(19) United States
(12) Patent Application Publication
(10) Pub. No.: US 2026/0253720 A1
(43) Pub. Date: Aug. 27, 2026

(54) INFORMATION COMMUNICATION SYSTEM, USER TERMINAL, COMPUTER CONTROL METHOD, AND COMPUTER PROGRAM

(71) Applicant: Onlinedoctor.com Co., Ltd., Minato-ku, Tokyo (JP)

(72) Inventor: Mikihiro SUZUKI, Minato-ku, Tokyo (JP)

(21) Appl. No.: 19/163,153

(22) PCT Filed: Dec. 19, 2023
(86) PCT No.: PCT/JP2023/045456

§ 371 Date: Sep. 8, 2025

(30) Foreign Application Priority Data
Mar. 8, 2023 (JP) 2023-035592

(51) Int. Cl. G16H 40/67; G06Q 10/02; G06V 40/16; G16H 10/60; G16H 15/00; G16H 50/30

(52) U.S. Cl. CPC G16H 40/67; G06Q 10/02; G06V 40/174; G16H 10/60; G16H 15/00; G16H 50/30

(57) ABSTRACT

The present invention provides an information communication system, a user terminal, a computer control method, and a computer program that make it possible to accurately and quickly acquire a change in vital information of a user even when the user is in a remote place. An information communication system is provided with: a user terminal serving as a detection means for detecting vital information of a user; an acquisition unit that acquires, online, the vital information detected by the user terminal; and a reporting unit that reports information related to the vital information acquired by the acquisition unit to a medical person terminal possessed by a prescribed relevant person.

### TECHNICAL FIELD

The present invention relates to an information communication system, a user terminal, a computer control method, and a computer program.

### BACKGROUND ART

As the operation of an online medical care for a patient residing in a remote area, there exists a conventional technology related to the online medical care, in which patient health information is collected on a doctor's computer via an intermediary server (for example, Patent Document 1).

### CITATION LIST

### Patent Document

Patent Document 1: Japanese Unexamined Patent Application, Publication No. 2022-16085

### DISCLOSURE OF THE INVENTION

### Problems to be Solved by the Invention

In a face-to-face medical examination, a doctor can accurately acquire vital information of a patient or the like by appropriately operating various medical devices, whereas in an online medical care, it is difficult for a doctor to accurately acquire vital information of a patient or the like because the doctor and the patient or the like are away from each other. In a case where a doctor and a medical examination user are at different places as in the online medical care or the like, it is preferable that the doctor quickly grasps a change in condition of the medical examination user.

The vital information of a subject is also an index indicating an emotion of the subject, and is expected to be used even in fields other than medical examination. However, in a case where the vital information of a subject is acquired in a studio or the like, the costs for the preparation of a room and the movement of the subject are necessary.

An object of the present invention, which has been made in such a background, is to provide an information communication system, a user terminal, a computer control method, and a computer program that make it possible to accurately and quickly acquire a change in vital information of a user even when the user is in a remote place.

### Means for Solving the Problems

In order to achieve the aforementioned object, an aspect of the present invention is to provide an information communication system including a detection unit that detects vital information of a user, an acquisition unit that acquires, online, the vital information detected by the detection unit, and a reporting unit that reports information related to the vital information acquired by the acquisition unit to a related-person terminal held by a predetermined related person.

An aspect of the present invention is to provide a user terminal including a detection unit that detects vital information of a user, a storage unit that records, offline, target information including at least one selected from the vital information, and information related to an amount injected, the number of injections, and meals of the user, and a transmission unit that transmits the target information to a server during an online time period.

An aspect of the present invention is to provide a computer control method including a detection step of detecting vital information of a user, an acquisition step of acquiring, online, the vital information detected in the detection step, and a reporting step of reporting information related to the vital information acquired in the acquisition step to a related-person terminal held by a predetermined related person.

An aspect of the present invention is to provide a computer program causing a computer to execute a detection step of detecting vital information of a user, an acquisition step of acquiring, online, the vital information detected in the detection step, and a reporting step of reporting information related to the vital information acquired in the acquisition step to a related-person terminal held by a predetermined related person.

### Effects of the Invention

According to the present invention, it is possible to provide an information communication system, a user terminal, a computer control method, and a computer program that make it possible to accurately and quickly acquire a change in vital information of a user even when the user is in a remote place.

### BRIEF DESCRIPTION OF THE DRAWINGS

FIG. 1 is a diagram illustrating an information communication system according to an embodiment of the present invention;

FIG. 2 is a block diagram illustrating a hardware configuration of an information processing apparatus according to the present embodiment;

FIG. 3 is a block diagram illustrating a functional configuration of the information communication system according to a first embodiment;

FIG. 4 is a schematic diagram illustrating a configuration of the information communication system according to the first embodiment;

FIG. 5 is a flowchart illustrating processing operation of the information communication system according to the first embodiment;

FIG. 6 is a block diagram illustrating a functional configuration of an information communication system according to a second embodiment;

FIG. 7 is a schematic diagram illustrating a configuration of the information communication system according to the second embodiment;

FIG. 8 is a block diagram illustrating a functional configuration of an information communication system according to a third embodiment;

FIG. 9 is a schematic diagram illustrating a configuration of the information communication system according to the third embodiment;

FIG. 10 is a block diagram illustrating a functional configuration of an information communication system according to a fourth embodiment;

FIG. 11 is a schematic diagram illustrating a configuration of the information communication system according to the fourth embodiment;

FIG. 12 is a schematic diagram illustrating a configuration of an information communication system according to a fifth embodiment;

FIG. 13 is a block diagram illustrating a functional configuration of an information communication system according to a sixth embodiment;

FIG. 14 is a schematic diagram illustrating a configuration of the information communication system according to the sixth embodiment; and

FIG. 15 is a schematic diagram illustrating a configuration of an online reservation system implemented by an information communication system according to a seventh embodiment.

### PREFERRED MODE FOR CARRYING OUT THE INVENTION

### System Configuration

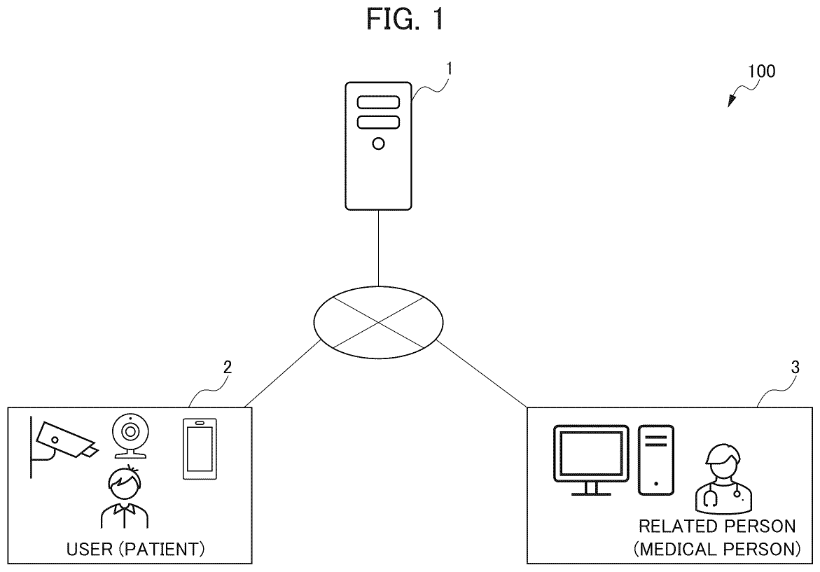

An embodiment of the present invention will be described below with reference to the drawings. First of all, an entire system configuration will be described. FIG. 1 is a diagram illustrating an information communication system 100 according to an embodiment of the present invention. An information processing apparatus 1 acquires vital information from a user who is in a remote area, and provides service for providing information related to the vital information to a predetermined related person. The user is, for example, a subject from which the vital information is acquired, such as a patient or the like who receives medical treatment from a medical person.

The information communication system 100 is implemented by the information processing apparatus 1 that transmits and receives various kinds of information to and from a user terminal 2 and a medical person terminal 3 through a communication network such as an Internet.

The information processing apparatus 1 is a computer that transmits and receives various kinds of information related to the vital information acquisition to and from the user terminal 2 and the medical person terminal 3, and functions as a server.

The user terminal 2 is a detection unit that acquires the vital information from the user. Examples of the user terminal 2 include a smartphone, a tablet, and a personal computer. The user terminal 2 may be a smart watch or a wearable device in which an apparatus that acquires the vital information is attached to a piece of clothing or the like.

The user terminal 2 can acquire the vital information through wired or wireless communication with an external detection device, for example. Examples of the external detection device include an image capturing apparatus such as a camera that captures an image of the user, a blood flow measuring apparatus such as a blood pressure measuring apparatus that acquires information related to a blood flow, an oxygen saturation measuring apparatus such as a pulse oximeter that measures a blood oxygen saturation, and a combination thereof. The image acquired by the image capturing apparatus is used to acquire heartbeat, respiration, blood pressure, blood oxygen, and the like by image analysis. The vital information acquired by an analysis process may be acquired using AI-based estimation such as machine learning. The information of heartbeat, respiration, blood pressure, blood oxygen, and the like may be analyzed from an image using AI-based estimation. The blood flow measuring apparatus is used by being attached to, for example, a wrist of the user. The oxygen saturation measuring apparatus is used by being attached to a finger of the user. Note that the user terminal 2 also includes an apparatus that acquires the above-described biometric information in a contactless manner (for example, acquires biometric information based on a face image of the user).

Note that the detection unit that acquires the vital information from the user is not limited to the user terminal 2. The detection unit may be a detection device such as an image capturing apparatus, a blood flow measuring apparatus, an oxygen saturation measuring apparatus, or a combination thereof. In this case, the vital information acquired by the detection device (detection unit) may be transmitted directly to the information processing apparatus 1 through the communication network without going through the user terminal 2.

The medical person terminal 3 is an information processing apparatus used by a utilizer (a predetermined related person) such as a medical person who utilizes the vital information of the user. The medical person terminal 3 is a tablet, a personal computer, a smartphone, or the like. The medical person is typically a doctor who performs medical treatment. The medical person terminal 3 receives the information related to the vital information of the user from the information processing apparatus 1. This enables the medical person to accurately and quickly acquire the vital information of the user who is in a remote area and the information related to the vital information.

### Hardware Configuration

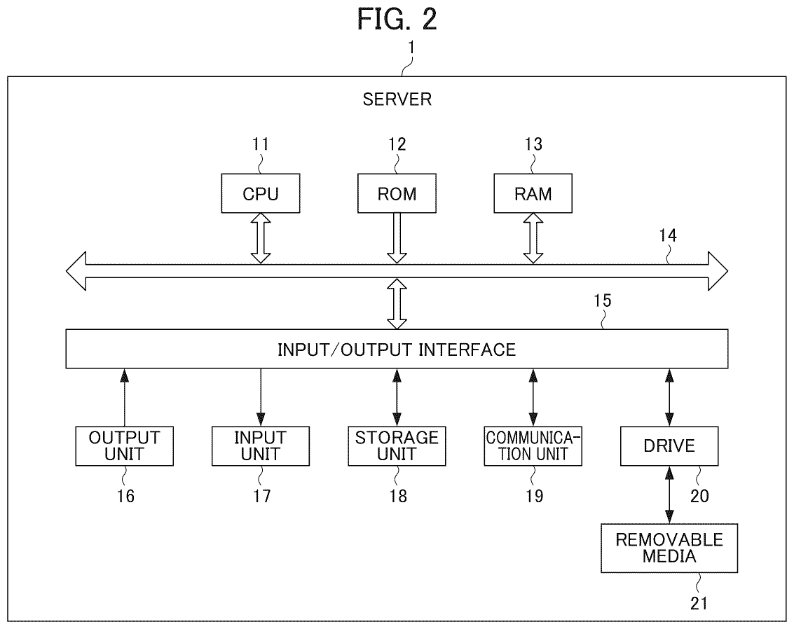

Next, an example of hardware forming the information processing apparatus 1 will be described. FIG. 2 is a block diagram illustrating a hardware configuration of the information processing apparatus according to the present embodiment. The information processing apparatus 1 includes a central processing unit (CPU) 11 as a processor, a read only memory (ROM) 12, a random access memory (RAM) 13, a bus 14, an input/output interface 15, an output unit 16, an input unit 17, a storage unit 18, a communication unit 19, and a drive 20.

The CPU 11 executes various processes according to a program stored in the ROM 12 or a program loaded into the RAM 13 from the storage unit 18. Items such as data necessary for the CPU 11 to execute the various processes are appropriately stored in the RAM 13. The CPU 11, the ROM 12, and the RAM 13 are connected to each other through the bus 14. The input/output interface 15 is also connected to the bus 14.

The output unit 16, the input unit 17, the storage unit 18, the communication unit 19, and the drive 20 are connected to the input/output interface 15. The output unit 16 includes a display, a speaker, or the like, and outputs various kinds of information as images or sound. The input unit 17 includes a keyboard, a mouse, or the like, and inputs various kinds of information. The storage unit 18 includes a hard disk, a dynamic random access memory (DRAM), or the like, and stores various kinds of data. The communication unit 19 performs communication between the other apparatuses through a network N including the Internet.

Removable media 21 such as a magnetic disk, an optical disk, a magneto-optical disk, or a semiconductor memory is appropriately mounted in the drive 20. A program read from the removable media 21 by the drive 20 is installed to the storage unit 18 as necessary. The removable media 21 can also store various kinds of data stored in the storage unit 18 in the same manner as the storage unit 18.

The hardware configuration described herein is merely one example. A computer described in the present embodiment, including the information processing apparatus 1, may have a configuration common to or different from the configuration in FIG. 2. The computer may be configured with two or more computers. Each of the user terminal 2 and the medical person terminal 3 in FIG. 1 is, for example, a smartphone, a tablet, or a personal computer that has a configuration similar to the hardware configuration illustrated in FIG. 2.

### First Embodiment

### <<Overview>>

Next, an in-home terminal care monitoring system implemented by the information communication system 100 having the above-described configuration will be described as a first embodiment. A problem of in-home terminal care is that an autopsy by police officers is not required if a doctor examines a person approaching death at home or at a nursing home, which is expected to expand rapidly in the future, within a certain period of time (in Japan, within 24 hours) prior to death (or after death), but if a person living alone died or a person died in the absence of a caregiver, an autopsy is required, which may impose psychological burden on family members of the person. In the present embodiment, there will be described the in-home terminal care monitoring system that monitors, in a contactless manner, an abnormality in the vital information in the terminal phase, and reports the abnormality to the doctor.

### <<Functional Configuration>>

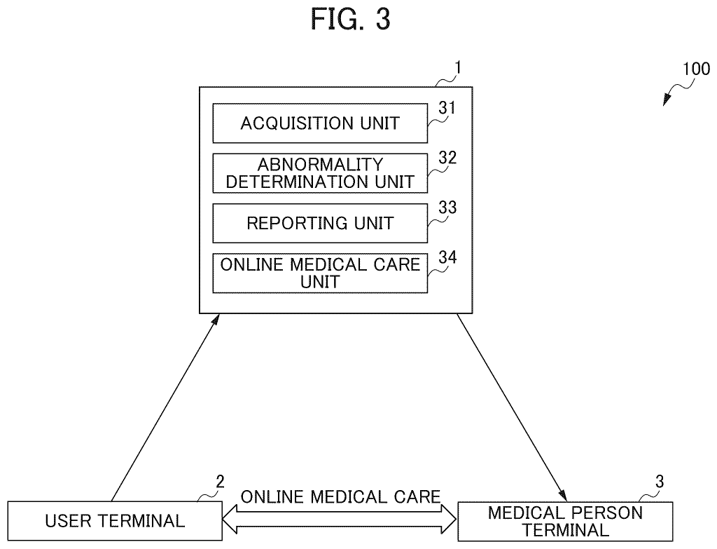

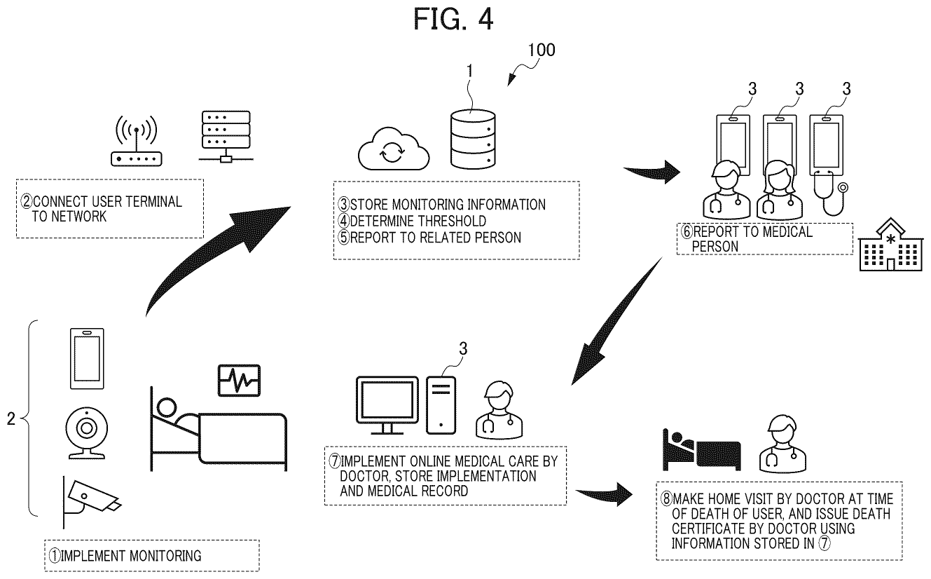

FIG. 3 is a block diagram illustrating a functional configuration of the information communication system 100 according to the first embodiment. FIG. 4 is a schematic diagram illustrating a configuration of the information communication system 100 according to the first embodiment.

The information processing apparatus 1 includes an acquisition unit 31, an abnormality determination unit 32, a reporting unit 33, and an online medical care unit 34 as functional units implemented on the processor (CPU 11).

First of all, in a first step, monitoring is performed in which the vital information of the user is acquired by the user terminal 2 functioning as a detection unit. As described above, the user terminal 2 is a smartphone (computer), a monitoring camera (image capturing apparatus), or the like, and acquires the vital information of an in-home user.

In a second step, the user terminal 2 is connected to the communication network, and transmits the monitored data to the information processing apparatus 1 functioning as a server. Each device that acquires the vital information may be connected to the network to transmit the vital information to the information processing apparatus 1.

In a third step, the acquisition unit 31 of the information processing apparatus 1 acquires and stores the vital information as a monitoring result from the user terminal 2. The acquisition unit 31 is an acquisition unit that communicates with the user terminal 2 through the communication network, acquires the vital information of the user, and stores the vital information in the storage unit 18.

In a fourth step, the abnormality determination unit 32 of the information processing apparatus 1 determines presence or absence of an abnormality in the vital information acquired by the acquisition unit 31. The abnormality determination unit 32 is a determination unit that determines whether the vital information satisfies a predetermined condition. The predetermined condition is, for example, that the blood pressure indicated by the vital information is a predetermined threshold or less, that the apnea duration indicated by the vital information exceeds a predetermined threshold, that the oxygen saturation indicated by the vital information is a predetermined threshold or less, that the arrhythmia indicated by the vital information exceeds a predetermined threshold, or the like.

In a fifth step, the reporting unit 33 transmits information based on the determination result of the abnormality determination unit 32 to the related-person terminal held by a predetermined related person such as a doctor in charge, a nurse, an emergency medical service, a care worker in charge, an intensive care unit (ICU) staff, or a family carer. The reporting unit 33 is a reporting unit that reports the information related to the vital information to the medical person terminal 3 serving as the related-person terminal held by the predetermined related person.

In a sixth step, the medical person terminal 3 receives the determination result of the abnormality determination unit 32 from the reporting unit 33 of the information processing apparatus 1. This makes it possible to send the determination result to the doctor in charge, the nurse, the emergency medical service, the care worker in charge, the ICU, and the like, and report the abnormality of the user to the related person.

In a seventh step, the online medical care unit 34 provides a video phone function for allowing the doctor to perform, online, medical care on the user using the medical person terminal 3. Alternatively, the online medical care unit 34 sends connection information such as an address for performing video phone to the doctor (medical person terminal 3) and the user (user terminal 2) who receives a medical examination. The online medical care unit 34 is an online medical care unit that executes a process for performing online medical care on the user. The online medical care unit 34 allows the doctor to perform the online medical examination based on the vital information every time an error occurs (an abnormality is determined). The process for performing the online medical care may be, for example, the process of sending an instruction to perform the online medical care to the medical person terminal 3, or the process of setting a schedule of the online medical care.

The online medical care unit 34 executes a process in which the doctor stores, from the medical person terminal 3, results of medical examination, a diagnosis, and a medical record by means of the online medical care or the like. The online medical care unit 34 executes the process for performing the online medical care again if the vital information of the user suddenly changes again after indicating revival of the user.

In an eighth step, the doctor makes a home visit at the time of death of the user, and issues a death certificate using medical record information stored by the online medical care. This makes it possible to implement a mechanism in which at the time of death of the user, the visiting doctor views the past medical examination information and can write the death certificate without an autopsy by police officers. Furthermore, the online medical care unit 34 may specify a doctor in a status of being capable of preparing a virtual waiting room constructed on the network as a doctor in charge of the online medical care.

The online medical care unit 34 executes the process for performing the online medical care based on a predetermined schedule in addition to the case where the vital information is satisfied the predetermined condition. The predetermined schedule is preferably scheduled to perform the online medical care before 24 hours have elapsed since the previous online medical care.

### <<Processing Contents>>

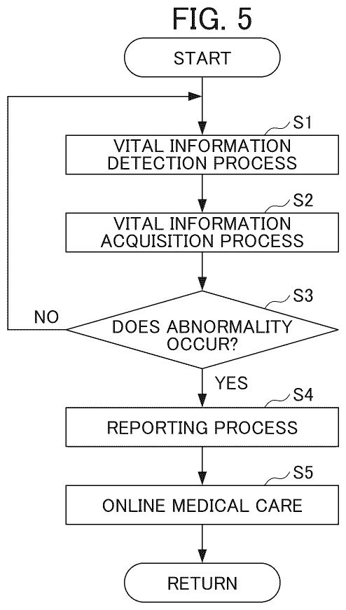

Next, a flow of processing of the information communication system 100 will be described with reference to FIG. 5. FIG. 5 is a flowchart illustrating processing operation of the information communication system 100 according to the first embodiment.

In step S1, the user terminal 2 executes a process of detecting the vital information of the user. The detection process corresponds to the implementation of the monitoring in FIG. 3.

In step S2, the acquisition unit 31 of the information processing apparatus 1 executes an acquisition process of acquiring the vital information acquired by the user terminal 2 through the communication network, and storing the acquired vital information in the storage unit 18. The acquisition process corresponds to the storage of the monitoring information in FIG. 3.

In step S3, the abnormality determination unit 32 of the information processing apparatus 1 executes a determination process of determining whether the acquired vital information satisfies a predetermined condition (abnormality condition). The determination process corresponds to the determination of the threshold in FIG. 3. If the predetermined condition is not satisfied, the abnormality determination unit 32 returns the process to the detection process (step S1; No), and if the predetermined condition is satisfied, the process proceeds to step S4 (step S3; Yes).

In step S4, the reporting unit 33 of the information processing apparatus 1 reports the information that the abnormality condition is satisfied (information related to the vital information) to the medical person terminal 3 of the doctor in charge who is a predetermined related person.

In step S5, the online medical care unit 34 executes the process for causing the doctor to perform the online medical care on the user who has the vital information satisfying the abnormality condition. This makes it possible to execute the online medical care on the user who has the vital information indicating an abnormality. After the process in step S5, the process returns to step S1, and the monitoring of the vital information is restarted.

### <<Advantageous Effects of the Present Embodiment>>

As described above, the information communication system 100 of the first embodiment includes the user terminal 2 serving as the detection unit that detects the vital information of the user, the acquisition unit 31 serving as the acquisition unit that acquires, online, the vital information detected by the user terminal 2, and the reporting unit 33 serving as the reporting unit that reports the information related to the vital information acquired by the acquisition unit 31 to the medical person terminal 3 held by the predetermined related person. This makes it possible for the predetermined related person such as a doctor to accurately and quickly acquire a change in vital information of the user even when the user is in a remote place.

The information communication system 100 of the first embodiment further includes the online medical care unit 34 that executes a process for performing online medical care on the user when the information related to the vital information is reported to the medical person terminal 3 by the reporting unit 33 in response to the fact that the vital information satisfies the predetermined condition. This makes it possible to provide a pseudo nurse station function to a family member, a doctor in charge, and a care worker in charge. In addition, a camera in a bedroom in which the user is present is configured as a primary detection device, and a terminal for a family member or a care worker is configured as a secondary detection device, so that “only” necessary information can be reported to the doctor at the necessary timing. A plurality of doctors are on the network, and therefore, even in such a case as orthostatic dysregulation (OD), anyone in Japan can determine the OD. An autopsy by police officers is not required if a doctor examines a person approaching death at home or at a nursing home, which is expected to expand rapidly in the future, within a certain period of time (in Japan, within 24 hours) prior to death, but if a person living alone died or a person died in the absence of a caregiver, an autopsy is required, which may impose psychological burden on family members of the person. In this regard, according to the configuration of the present embodiment, the medical care can be performed online within a certain period of time, making it possible to reduce psychological burden on family members.

### Second Embodiment

### <<Overview>>

Next, a second embodiment will be described in which a system that monitors, in a contactless manner, an abnormality in vital information of a user and reports the abnormality to a doctor is implemented by an information communication system 100a having a configuration similar to that of the first embodiment. In recent years, in the online medical care sites, a mechanism is desired in which the vital information of the user is acquired and stored in a contactless manner, and online real-time information or stored non-real-time information is conveyed to a doctor. An information communication mechanism is desired which, in a state in which a doctor is absent, makes it possible to detect an abnormality of a patient while taking care of the patient. The information communication system 100a of the second embodiment acquires, stores, or monitors the vital information of the user during online medical care in real time and in a contactless manner, and reports the vital information to the doctor. Note that, in the following description, like reference numerals are used to denote common or similar components to the components in the first embodiment, and their detailed descriptions may thus be omitted.

### <<Functional Configuration>>

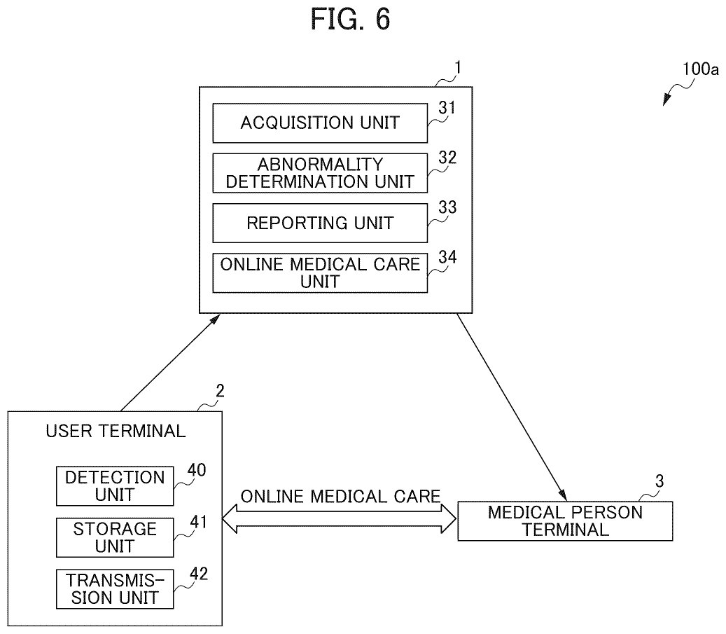

FIG. 6 is a block diagram illustrating a functional configuration of the information communication system 100a according to the second embodiment. As illustrated in FIG. 6, a user terminal 2 of the second embodiment includes a detection unit 40, a storage unit 41, and a transmission unit 42 as functional units implemented on the processor of the user terminal 2. Note that an information processing apparatus 1 has a configuration similar to that of the first embodiment.

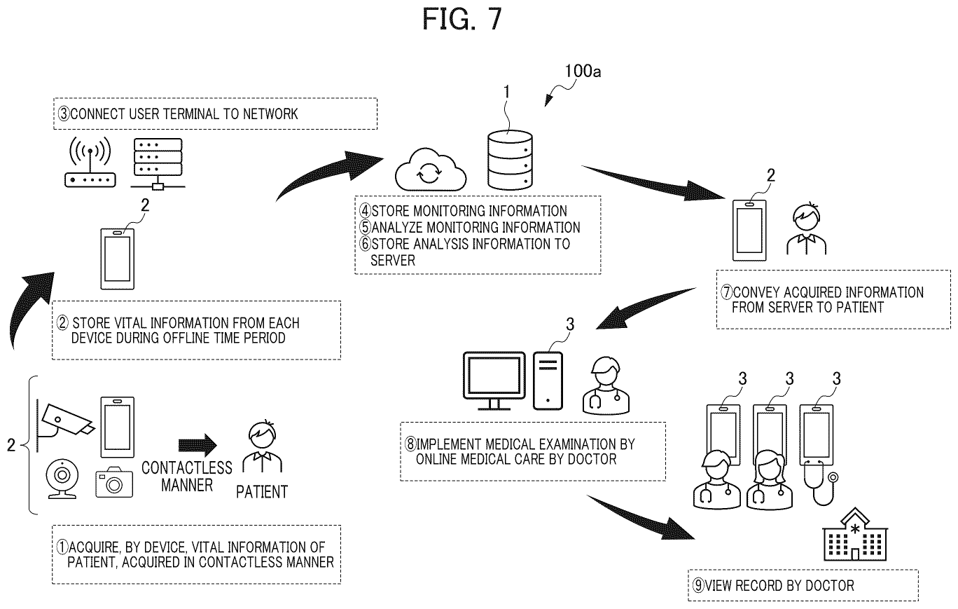

FIG. 7 is a schematic diagram illustrating a configuration of the information communication system 100a according to the second embodiment. First of all, in a first step, the vital information of a patient during online medical care is acquired by the detection unit 40 of the user terminal 2 which is a device configured to acquire the vital information in a contactless manner. The user terminal 2 is a device such as a smartphone, a tablet, or a personal computer. The detection unit 40 may have an image capturing function of the user terminal 2 itself, or an external device such as a web camera, a digital camera, or a monitoring camera that is communicable wirelessly or wiredly with the user terminal 2.

In a second step, the storage unit 41 of the user terminal 2 stores the vital information from each device in the user terminal 2 during an offline time period. Each device in this example is an external device such as a web camera, a digital camera, a monitoring camera, or various sensors. The vital information acquired by each device is wirelessly or wiredly transmitted to and stored in the user terminal 2 during the offline time period in which the online medical care is not performed. In a case where the user terminal 2 itself acquires the vital information, the vital information is stored in the user terminal 2. The storage unit 41 is a storage unit that wirelessly or wiredly stores the detected vital information of the user in the user terminal 2.

In a third step, the transmission unit 42 of the user terminal 2 is connected to the network and transmits data such as the acquired vital information to the information processing apparatus 1. The data is transmitted directly to the information processing apparatus 1 functioning as a server. The transmission unit 42 is a transmission unit that conveys the information stored in the user terminal 2 to an online server and stores it in the server.

In a fourth step, an acquisition unit 31 of the information processing apparatus 1 acquires and stores the vital information as a monitoring result from the user terminal 2. In a fifth step, an abnormality determination unit 32 analyzes the information of heartbeat, respiration, blood pressure, blood oxygen, and the like based on the monitoring information. The analysis is performed by, for example, AI or a predetermined algorithm. In a sixth step, the abnormality determination unit 32 stores the analysis information of the abnormality determination unit 32 in the storage unit 18. In a seventh step, a reporting unit 33 of the information processing apparatus 1 conveys the acquired analysis information to the user terminal 2 of the patient. In an eighth step, an online medical care unit 34 executes a process for performing online medical care. In the online medical care, a process of storing a medical record of the performed medical examination is executed. In a ninth step, the doctor or the like confirms the medical record of the online medical care or the like using a medical person terminal 3.

### <<Advantageous Effects of the Present Embodiment>>

As described above, the information communication system 100a of the second embodiment includes the storage unit 41 that stores the detected vital information of the user in the user terminal 2, and the transmission unit 42 that conveys the information stored in the user terminal 2 to the online server, in addition to the components of the first embodiment.

This makes it possible to implement a mechanism in which the vital information of the user is acquired and stored in a contactless manner, and on-line real-time information or stored non-real-time information is conveyed to the doctor. The mechanism makes it possible to, in a state in which the doctor is absent, detect an abnormality of the patient while taking care of the patient.

Note that, in the second embodiment, each storing process in the second, fourth, and sixth steps can be omitted, and the vital information and its analysis result can be transmitted directly to the user terminal 2 and the medical person terminal 3.

### Third Embodiment

### <<Overview>>

In recent years, a mechanism is desired in which transition information of a blood glucose level (by which an insulin injection unit is determined) of a diabetic patient to be always monitored is reliably conveyed to a doctor and a patient himself/herself. Then, a third embodiment will be described in which a system that monitors, in a contactless manner, an abnormality in vital information of a user who is a diabetic patient and reports the abnormality to a doctor is implemented by an information communication system 100b having a configuration similar to that of the second embodiment.

### <<Functional Configuration>>

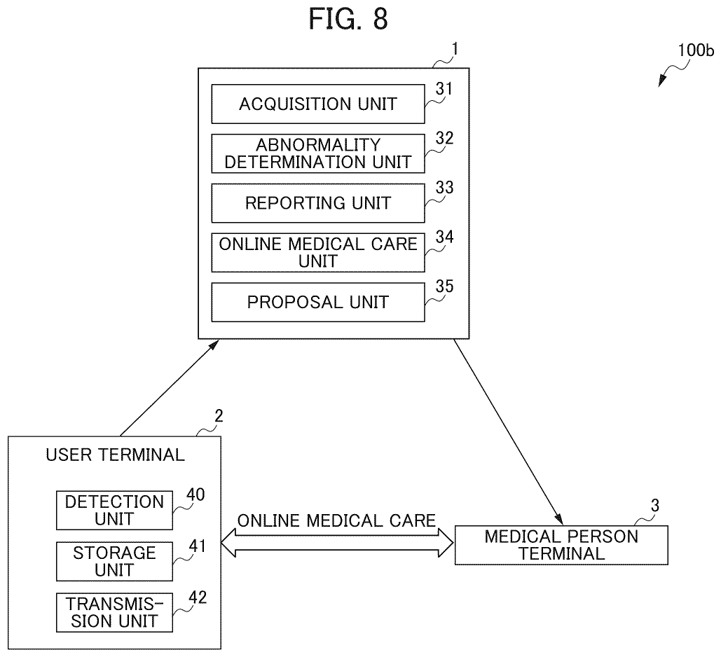

FIG. 8 is a block diagram illustrating a functional configuration of the information communication system 100b according to the third embodiment. As illustrated in FIG. 8, an information processing apparatus 1 of the third embodiment further includes a proposal unit 35 that proposes insulin dose, in addition to the components of the information processing apparatus 1 of the second embodiment.

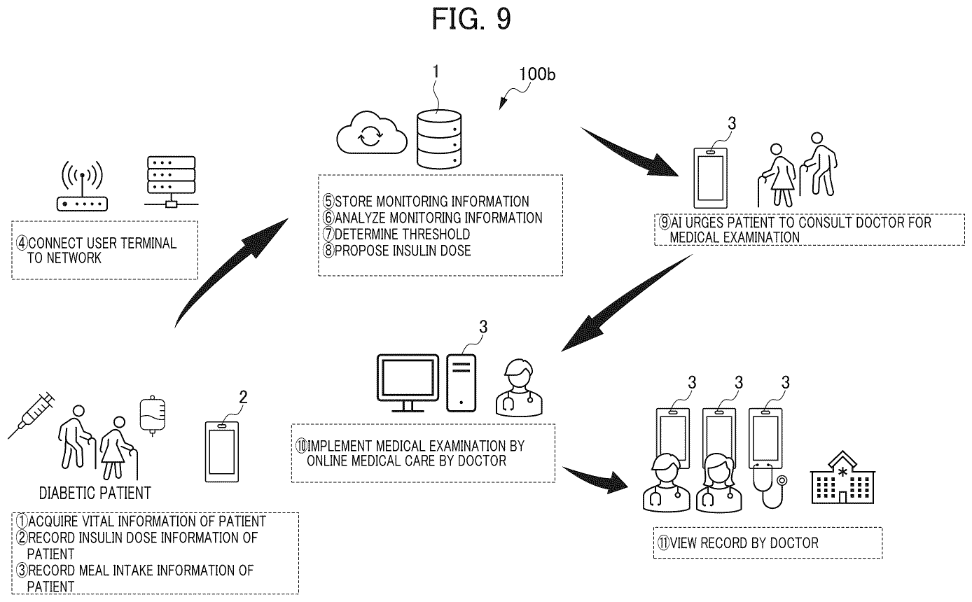

FIG. 9 is a schematic diagram illustrating a configuration of the information communication system 100b according to the third embodiment. As illustrated in FIG. 9, in a first step, the vital information of the diabetic patient is acquired by a user terminal 2. In a second step, the insulin dose information of the diabetic patient is recorded by the user terminal 2. In a third step, the meal intake information (information related to meals) of the patient is recorded by the user terminal 2. The meal intake information is acquired by, for example, capturing an image of a target meal using the user terminal 2. The meal intake information may be acquired by image analysis or may be acquired by user input. Note that the insulin dose information and the meal intake information may be acquired from an external device other than the user terminal 2. Note that the meal intake information (information related to meals) is not limited to particular information, but preferably includes at least one selected from, for example, the meal contents, the number of meals, and nutrients of meals.

In a fourth step, a transmission unit 42 of the user terminal 2 is connected to the network and transmits target information such as the acquired vital information to the information processing apparatus 1. The target information includes the insulin dose information of the diabetic patient, the meal intake information of the patient, and the like. In this way, the user terminal 2 has a storage area in which an amount injected, the number of injections can be recorded offline, and a function of transmitting the stored information to a server during an online time period.

In a fifth step, an acquisition unit 31 of the information processing apparatus 1 acquires and stores the vital information as a monitoring result from the user terminal 2. In a sixth step, an abnormality determination unit 32 analyzes the information of heartbeat, respiration, blood pressure, blood oxygen, and the like based on the monitoring information. In a seventh step, the abnormality determination unit 32 determines an abnormality based on the vital information. In this example, the abnormality determination unit 32 determines the quality of the nutrient balance based on the meal record of the user.

In an eighth step, the proposal unit 35 proposes an appropriate insulin dose from the patient data such as the vital information. The proposal unit 35 may propose appropriately nutrient-balanced meals. In a ninth step, a reporting unit 33 transmits, to the user terminal 2, the information urging the user to consult a doctor for a medical examination. For the determination as to whether to urge the user to consult a doctor for a medical examination, AI may be used. This makes it possible to urge the doctor to perform a medical examination on the diabetic patient. In a tenth step, an online medical care unit 34 executes a process for performing online medical care. In an eleventh step, the doctor or the like confirms the medical record of the online medical care or the like using a medical person terminal 3.

### <<Advantageous Effects of the Present Embodiment>>

As described above, in the third embodiment, the acquisition unit 31 acquires the patient data including at least one selected from an amount of insulin injection, the number of insulin injections, and meals of the user, and the reporting unit 33 reports, to the user, at least one selected from the insulin dose and the meals reflecting nutrient balance based on the patient data. The user terminal 2 includes a detection unit 40 that detects the vital information of the user, a storage unit 41 that records, offline, the target information including at least one selected from the vital information, and information related to an amount injected, the number of injections, and meals of the user, and a transmission unit 42 that transmits the target information to a server during an online time period.

An amount of insulin injection is instructed by the doctor, but the patient himself/herself can determine the injection unit amount by measuring his/her blood glucose level by himself/herself when becoming more accustomed to the injection experience, making it impossible for the doctor to accurately grasp the amount injected and the number of injections of the patient. It is difficult to appropriately communicate between the doctor and the patient in the medical examination for a short time.

In order to accurately grasp the blood glucose level of the patient, the patient needs to record the blood glucose levels by handwriting (before each meal, 2 hours after a meal, and at a low blood glucose level), and needs to show the records to the doctor, but the recording is troublesome for the patient, so that the patient may abandon the recording. In this regard, according to the configuration of the present embodiment, the history of the injections remains, making it possible to determine whether the usage is appropriate and provide guidance. It is possible to reliably convey the transition information of blood glucose levels and the like to the doctor and the patient himself/herself using a portable terminal such as a smartphone of the patient himself/herself without relying on an implantable type device. In particular, it is possible to reduce medical questionnaire omission and intentional hiding by the diabetic patient himself/herself. In the case of young patients, the occurrence of a low blood glucose level is unavoidable, which enables excessive insulin injection to be avoided. It is also conceivable that the patient customizes the injection unit by himself/herself under the guidance of the doctor. The doctor exclusively infers whether the patient can control blood glucose using HbA1C, but the HbA1C is the average value of blood glucose levels over a period of one month, making it impossible to determine that low blood glucose levels and high blood glucose levels are mixedly present, so that the doctor cannot strictly determine an appropriate unit amount. Also in this regard, according to the configuration of the present embodiment, the amount injected is monitored, making it possible to accurately grasp a high blood glucose level after a meal and a low blood glucose level before a meal and safely determine the insulin amount. Furthermore, a dietetic therapy is important in the medical care of diabetes, but the patient can eat a lot as long as the patient takes insulin injection, so that the patient spoils himself/herself. With this regard, an image of the meal is captured by a camera of the user terminal 2, so that appropriate caloric values of glucide and the like can be grasped.

Note that the configuration of the third embodiment is effective for the user who controls blood glucose levels, such as a person on a diet, a body builder, or a boxer in addition to the diabetic patient.

### Fourth Embodiment

### <<Overview>>

In recent years, a mechanism is desired which assists determination as to whether emergency medical service or the like is necessary, based on the vital information in which a normal value differs depending on the patient (for example, a determination as to whether a “blood pressure of 190” is accommodated to the emergency medical service based on transition from the normal blood pressure). Then, a fourth embodiment will be described in which a system that assists determination as to whether emergency medical service or the like is necessary, based on the vital information in which a normal value differs depending on the patient is implemented by an information communication system 100c having a configuration similar to that of the third embodiment. The determination as to whether emergency medical service or the like is necessary is, for example, a determination as to whether a “blood pressure of 190” is accommodated to the emergency medical service based on transition from the normal blood pressure.

### <<Functional Configuration>>

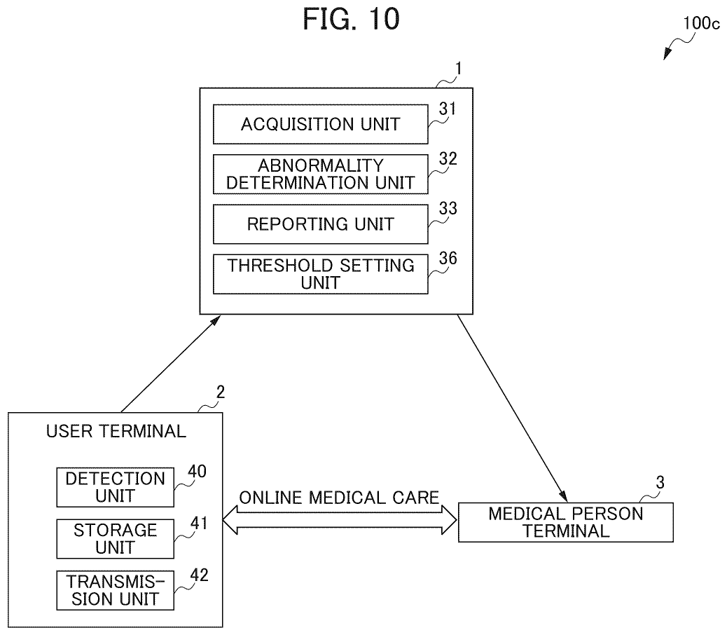

FIG. 10 is a block diagram illustrating a functional configuration of the information communication system 100c according to the fourth embodiment. As illustrated in FIG. 10, an information processing apparatus 1 of the fourth embodiment further includes a threshold setting unit 36 that sets a threshold for determining an abnormality, in addition to the components of the information processing apparatus 1 of the second embodiment.

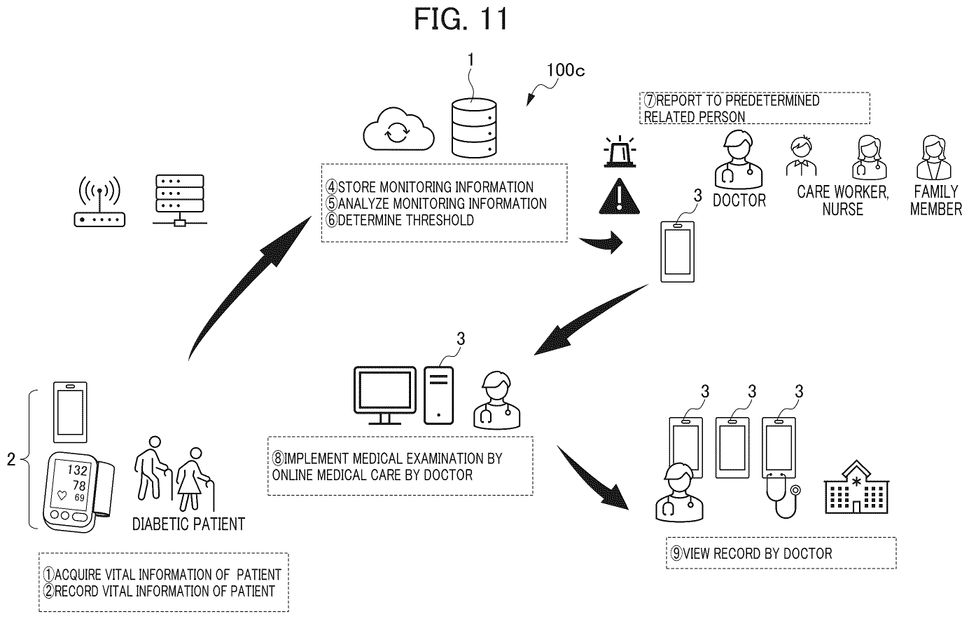

FIG. 11 is a schematic diagram illustrating a configuration of the information communication system 100c according to the fourth embodiment. In a first step, the vital information of a hypertensive patient is acquired by a user terminal 2. The blood pressure is acquired by, for example, a sphygmomanometer attached to an arm of the user. The measurement result of the sphygmomanometer may be input as the blood pressure by the user or the user terminal 2 may wirelessly or wiredly acquire the blood pressure from the sphy gmomanometer. Alternatively, the blood pressure may be acquired by image analysis of the user terminal 2. In a second step, the vital information including the blood pressure of the hypertensive patient is recorded by the user terminal 2.

In a third step, a transmission unit 42 of the user terminal 2 is connected to the network and transmits data such as the acquired vital information including the blood pressure to the information processing apparatus 1. In a fourth step, an acquisition unit 31 of the information processing apparatus 1 acquires the vital information as a monitoring result from the user terminal 2.

In a fifth step, an abnormality determination unit 32 analyzes the information of heartbeat, respiration, blood pressure, blood oxygen, and the like based on the monitoring information. The threshold setting unit 36 sets a threshold based on the analysis result. The threshold setting unit 36 is a threshold setting unit that sets an abnormality determination threshold based on the past transition of the vital information of the user. The threshold may be set by means of AI such as machine learning based on the past transition of the user or may be set based on a predetermined algorithm. When the vital information deviates from the threshold set based on the past transition of the value, it is estimated that an abnormality occurs in the user.

In a sixth step, the abnormality determination unit 32 determines an abnormality based on the threshold set by the threshold setting unit 36. Since the threshold is set based on the past transition of the vital information of the user, even with the same blood pressure value, the user may be determined to in an abnormal condition requiring emergency transportation or may be determined not to require emergency transportation.

In a seventh step, a reporting unit 33 reports the information related to the vital information to the medical person terminal 3 serving as the related-person terminal held by the predetermined related person. In this example, the fact that an abnormality occurs is reported to the doctor by the reporting unit 33.

In an eighth step, an online medical care unit 34 executes a process for performing online medical care. In a ninth step, the doctor or the like confirms the medical record of the online medical care or the like using a medical person terminal 3.

### <<Advantageous Effects of the Present Embodiment>>

As described above, the information communication system 100c of the fourth embodiment further includes the threshold setting unit 36 that sets an abnormality determination threshold based on the past transition of the vital information of the user, and the reporting unit 33 reports the abnormality to the predetermined related person if the vital information exceeds the threshold for the user.

This makes it possible to reduce unnecessary emergency medical service requests while urging request of the emergency medical service for the patient with actually high emergency level. The blood pressure of the patient with orthostatic dysregulation or the like rapidly drops to 70 or the like, and the patient may fall. With this regard, according to the fourth embodiment, since the threshold is set based on the past transition, the abnormality of the user can be determined even in such a case. It is possible to use for estimating a cause in the event of the abnormality in a combination with a Holter electrocardiogram. In a day service facility or the like, the bathing service or the like is currently provided according to a certain blood pressure threshold regardless of the normal blood pressure of the patient himself/herself, but the condition of the patient is clarified by the system of the fourth embodiment, helping to determine whether the service can be utilized. The system can be utilized in a wide range of fields in a school infirmary or the like in addition to a home.

### Fifth Embodiment

### <<Overview>>

In recent years, an information communication mechanism is desired which can detect an abnormality while watching the vital information of the user. Then, a fifth embodiment will be described in which a system that acquires and stores, or monitors, in a contactless manner, an abnormality in vital information of a user during the web conference, and reports the abnormality to a doctor is implemented by an information communication system 100d having a configuration similar to that of the second embodiment.

### <<Functional Configuration>>

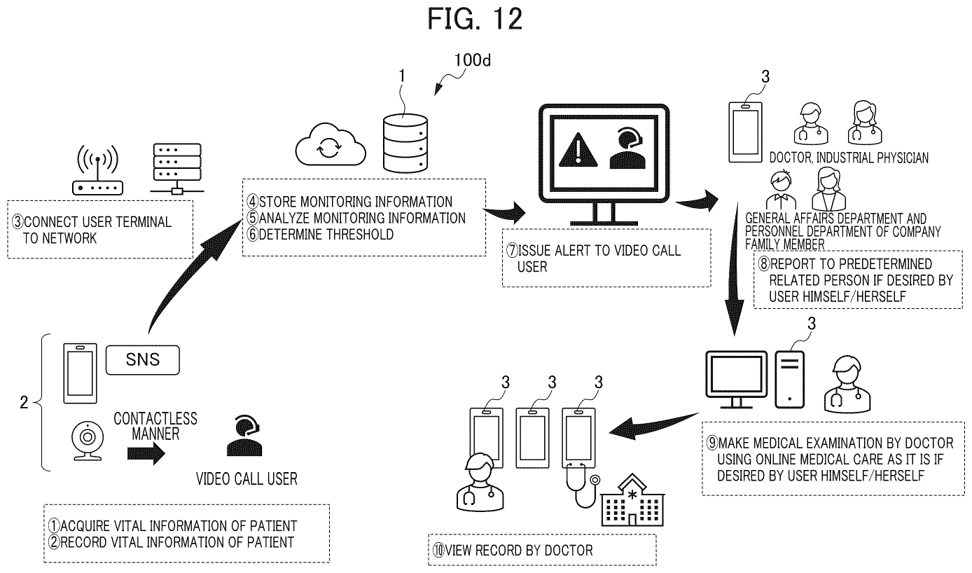

FIG. 12 is a schematic diagram illustrating a configuration of the information communication system 100d according to the fifth embodiment. In a first step, the vital information during a video call is acquired by a user terminal 2. The vital information includes sympathetic nerves, parasympathetic nerves, and the like in addition to heartbeat, respiration, blood pressure, and blood oxygen. The sympathetic nerves and the parasympathetic nerves can be acquired by heart rate variability analysis using, for example, an electrocardiogram and a pulse wave. The video call is implemented by, for example, a conferencing system such as ZOOM (registered trademark) or Google Meet, and a social networking service (SNS) such as LINE (registered trademark) or Messenger. In a second step, the vital information is recorded by the user terminal 2.

In a third step, a transmission unit 42 of the user terminal 2 is connected to the network and transmits data such as the acquired vital information to the information processing apparatus 1. In a fourth step, an acquisition unit 31 of the information processing apparatus 1 acquires the vital information as a monitoring result from the user terminal 2. In a fifth step, an abnormality determination unit 32 analyzes the information of heartbeat, respiration, blood pressure, blood oxygen, sympathetic nerves, parasympathetic nerves, and the like based on the monitoring information. In a sixth step, the abnormality determination unit 32 of the information processing apparatus 1 determines presence or absence of an abnormality in the vital information acquired by the acquisition unit 31.

In a seventh step, a reporting unit 33 issues an alert to the user who is a video call user. This alert is reported when the abnormality determination unit 32 determines that an abnormality occurs. The alert may be reported based on the vital information acquired within a predetermined short period of time, or may be reported based on the accumulated vital information of the user. The alert is displayed on, for example, the user terminal 2 held by the user.

In an eighth step, if desired by the user himself/herself, the reporting unit 33 reports the information related to the vital information to a related-person terminal held by a predetermined related person. In this example, the reporting unit 33 reports the determination result of the abnormality determination unit 32 and the occurrence of an abnormality to the terminals of the general affairs department and the personnel department of the company, and the family members in addition to the medical person terminal 3 of the doctor or the industrial physician. This makes it possible to set the reporting to the related person, if desired by the user himself/herself.

In a ninth step, if desired by the user himself/herself, an online medical care unit 34 executes a process for performing online medical care. For example, the desire to receive the online medical care is reported to the information processing apparatus 1 through the user terminal 2. In a tenth step, the doctor or the like confirms the medical record of the online medical care or the like using a medical person terminal 3.

### <<Advantageous Effects of The Present Embodiment>>

As described above, a detection unit 40 of the user terminal 2 of the fifth embodiment detects (acquires) the vital information of the user during the web conference.

This makes it possible to determine the abnormality and condition of the user during the web conference such as a video conference. Even in the state in which the web conference in the company, the video chat with the family member or the friend, or the like is performed, an action in an unlikely event can be taken. When the parasympathetic nerves are included in the vital information, it is possible to monitor dozing during the web conference, monitor the side job, and estimate the productivity of the conference. The reporting includes the reporting to the user himself/herself or to the related person. The reporting to the related person may be set if desired by the user himself/herself. When the alert can be issued on the video call screen, the fact that the abnormality occurs can be quickly conveyed to the user.

### Sixth Embodiment

### <<Overview>>

In recent years, a content delivery mechanism is desired which can monitor excitement or emotion of the user while watching the vital information of the user. Then, a sixth embodiment will be described which implements a system used for content delivery that can monitor excitement or emotion of the user while watching the vital information of the user.

### <<Functional Configuration>>

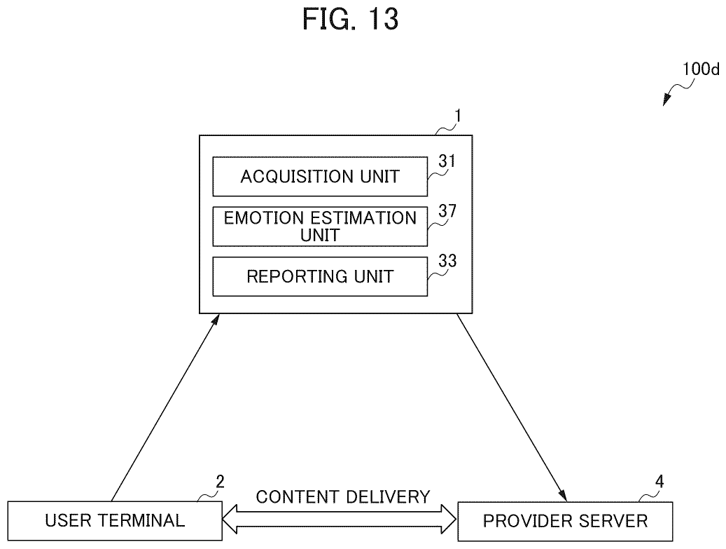

FIG. 13 is a block diagram illustrating a functional configuration of an information communication system 100e according to the sixth embodiment. As illustrated in FIG. 13, the information communication system 100e includes an information processing apparatus 1, a user terminal 2, and a provider server 4.

The information processing apparatus 1 is a server that acquires the vital information when the user uses the contents provided by a content delivery system producer, and provides service that provides the analysis result based on the vital information to the content delivery system producer. The contents provided by the content delivery system producer include videos, music, games, and the like.

The user terminal 2 is a terminal held by the user who views the content, and acquires the vital information of the user. The user may view the content using the user terminal 2, or may view the content using a device such as television or another personal computer.

The provider server 4 is a computer managed by a provider that delivers the contents to the user. The provider server 4 is, for example, a server that manages, for example, the user information and the vital information. The provider server 4 acquires the information related to the vital information of the user when the content is used via the information processing apparatus 1. Note that the provider server 4 may be a server that delivers the content, but is not limited to the server that delivers the content.

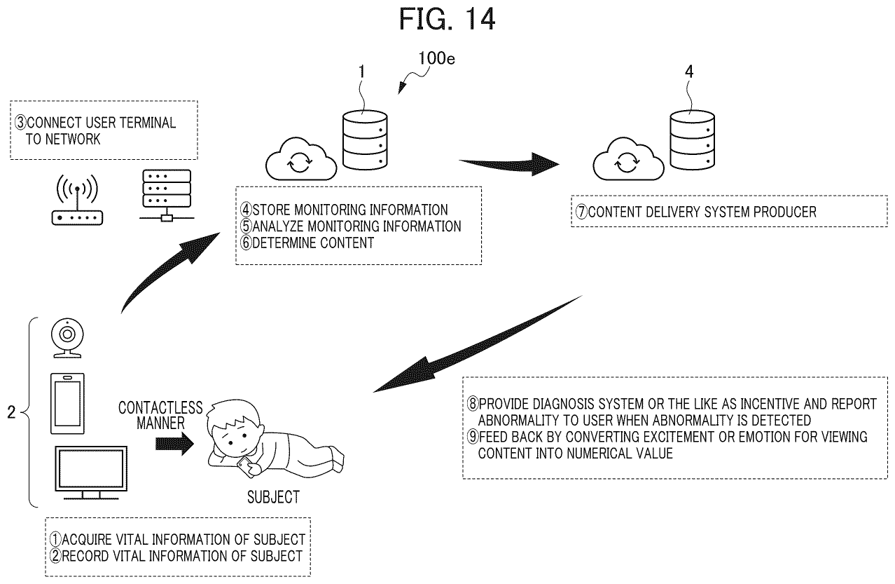

The information processing apparatus 1 includes an acquisition unit 31, an emotion estimation unit 37, and a reporting unit 33 as functional units implemented on the processor. Each functional unit of the information processing apparatus 1 will be described with reference to FIG. 14. FIG. 14 is a schematic diagram illustrating a configuration of the information communication system 100e according to the sixth embodiment.

First of all, in a first step, the vital information during use of the content is acquired by the user terminal 2. The vital information includes heartbeat, respiration, blood pressure, blood oxygen, sympathetic nerves, parasympathetic nerves, and the like. The sympathetic nerves and the parasympathetic nerves can be acquired by heart rate variability analysis using, for example, a pulse wave. In a second step, the vital information is recorded by the user terminal 2.

In a third step, a transmission unit 42 of the user terminal 2 is connected to the network and transmits data such as the acquired vital information to the information processing apparatus 1. In a fourth step, an acquisition unit 31 of the information processing apparatus 1 acquires the vital information as a monitoring result from the user terminal 2. In a fifth step, the emotion estimation unit 37 analyzes emotional ups and downs of the user when viewing the content based on the monitoring information. In a sixth step, the emotion estimation unit 37 determines the content quality based on the analyzed emotional ups and downs of the user. The determination of content quality may be performed based on, for example, the threshold determined by pre-experiment or based on the machine learning model such as AI. In a seventh step, the reporting unit 33 transmits, to the provider server 4, the analysis result indicating the emotion of the user when viewing the content and the information indicating the content quality. This enables the user information when the user views the content to be accumulated in the provider server 4.

In an eighth step, the provider server 4 provides a diagnosis system, or the like as an incentive to the user terminal 2. In the diagnosis system, for example, an abnormality of the user that is detected when the user views the content is reported to the user terminal 2. In a ninth step, the provider server 4 feeds back, to the user terminal 2, the information in which the excitement or emotion for the viewing content is converted into a numerical value.

### <<Advantageous Effects of the Present Embodiment>>

As described above, the information communication system 100e of the sixth embodiment includes the user terminal 2 serving as the detection unit that detects the vital information of the user, the acquisition unit 31 serving as the acquisition unit that acquires, online, the vital information detected by the user terminal 2, the reporting unit 33 serving as the reporting unit that reports the information related to the vital information acquired by the acquisition unit 31 to the provider server 4 held by the predetermined related person, and the emotion estimation unit 37 that estimates the emotion of the user based on the vital information.

This makes it possible to feed back, to the content delivery system producer, the emotion of the user when using the content, estimated based on the vital information. An impressed scene of a movie can be extracted, and interest in and satisfaction with the content can be estimated. The information of the emotion or the like of user for the content can be utilized for evaluating a creator of the content, or determining the charging for the advertisement rate.

### Seventh Embodiment

### <<Overview>>

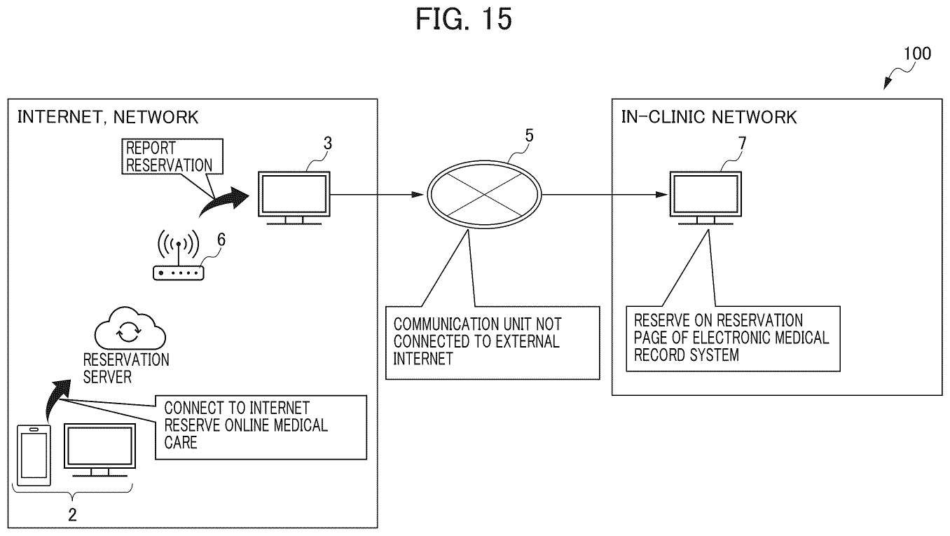

In an on-premises type electronic medical record system in which an information system such as a so-called server, or software is usually installed and operated within a facility that manages the system by itself, a server and a client terminal are installed on the network completely disconnected from the Internet as security measures. Therefore, the doctor or medical clerical staff needs to manually input the information reserved by the reservation system on the home page to cooperate with the electronic medical records for the doctor. Therefore, a mechanism is desired in which the doctor reflects the reservation to a reservation time slot of the electronic medical record system for the doctor through the network in response to reception of the information of the patient desiring to visit the hospital. Then, an online reservation system that solves the problem will be described with reference to FIG. 15.

### <<Functional Configuration>>

FIG. 15 is a schematic diagram illustrating a configuration of an online reservation system implemented by an information communication system 100 according to a seventh embodiment. The online reservation system implemented by the information communication system 100 according to the seventh embodiment can be applied to any of the above-described first to sixth embodiments.

As illustrated in FIG. 15, the online reservation system implemented by the information communication system 100 includes a user terminal 2 and a medical person terminal 3 in the Internet network, a communication unit 5, and a reservation terminal 7 in an in-clinic network. Note that the information processing apparatus 1 is not illustrated in FIG. 15.

The user terminal 2 is connected to the Internet by, for example, a communication line contracted by the user himself/herself or an external communication line. The user terminal 2 reserves the online medical care at the reservation server through the Internet. For the reservation, the doctor in charge, the date and time, and the like can be designated. The user can select the reservation of not only the online medical care but also the hospital visit.

The medical person terminal 3 is connected to the Internet through a router in the clinic. The medical person terminal 3 receives the reservation information reported from the reservation server through the router 6. The reservation information may be sent, for example, by e-mail desiring a predetermined hospital visit from a predetermined patient to a predetermined doctor

The communication unit 5 is a communication unit that is not connected to the external Internet. The communication unit 5 wirelessly or wiredly connects the medical person terminal 3 and the reservation terminal 7, for example. Since the communication unit 5 is not connected to the Internet, the information can be safely conveyed. The communication unit 5 is, for example, Bluetooth (registered trademark).

The reservation terminal 7 is a computer connected to the in-clinic network, and is a hospital visit reservation unit that registers the reservation information with a predetermined electronic medical record system. The in-clinic network is a secure network that is not connected to the Internet. The reservation terminal 7 constructs the electronic medical record system that manages the reservation.

The reservation terminal 7 acquires the reservation information from the medical person terminal 3 through the communication unit 5, and registers the reservation information with the electronic medical record system based on the reservation information. This makes it possible to register the schedule of the online medical care for the patient and the doctor.

For a process of reflecting the reservation in response to reception of the reservation information from the medical person terminal 3, an application programming interface (API) and a robotic process automation (RPA) operation can be used.

### <<Advantageous Effects of The Present Embodiment>>

As described above, the information communication system 100 of the seventh embodiment further includes the medical person terminal 3 serving as the reception unit that receives, from the user, the reservation information indicating a desire to receive a medical examination, and the reservation terminal 7 serving as the hospital visit reservation unit that registers the reservation information with the predetermined electronic medical record system through the predetermined communication unit 5 in response to reception of the reservation information without going through the Internet.

This makes it possible to automatically register the hospital visit reservation information with the electronic medical record system not connected to the Internet, from the medical person terminal 3 connected to the Internet. The online reservation system implemented in the seventh embodiment is a system that is generally used when the information connected to the Internet in addition to the reservation information is provided to another terminal on the network not connected to the Internet.

### <Others>

An embodiment of the present invention has been described above, but the present invention is not limited to the above-described embodiment. The present invention includes any alteration, improvement, and the like within the scope of achieving the purpose of the present invention.

The series of processes described above can be executed by hardware or software. In other words, the above-described functional configuration is only an example, and is not a particular limitation. That is, it is sufficient that the information processing apparatus includes functions capable of executing the series of processes described above as a whole, and the functional blocks which are used to achieve these functions are not particularly limited to the example described above. The locations of the functional blocks are not limited to particular locations, and can be arbitrary. For example, the functional blocks of the information processing apparatus may be transferred to other apparatuses, and the like. Conversely, the functional blocks of other apparatuses may be transferred to the server or the like. One functional block may consist of hardware alone, software alone, or a combination of the two.

When a series of processes is executed by software, the programs constituting the software are installed on a computer or other devices, from a network or recording medium. The computer may be a computer integrated in dedicated hardware. The computer may also be a computer capable of performing various functions by installing various programs, for example, a server as well as a general-purpose smartphone or personal computer.

The recording medium containing such a program includes not only a removable medium (not illustrated) that is distributed separately from the main unit of the apparatus in order to provide the program, but also a recording medium or the like pre-installed in the main unit of the apparatus. Since the program can be distributed through a network, the recording medium may be mounted on or accessible to a computer connected or connectable to the network.

In this specification, the steps of describing a program recorded on a recording medium include not only processes that are performed chronologically according to their sequence, but also processes that are performed in parallel or individually without necessarily being processed chronologically. In this specification, the term “system” refers to an overall apparatus including multiple apparatuses, multiple units, and the like.

### EXPLANATION OF REFERENCE NUMERALS

1 Information processing apparatus
2 User terminal
3 Medical person terminal
4 Provider server
7 Reservation terminal
31 Acquisition unit
32 Abnormality determination unit
33 Reporting unit
34 Online medical care unit
35 Proposal unit
36 Threshold setting unit
37 Emotion estimation unit
40 Detection unit
41 Storage unit
42 Transmission unit
100, 100a to 100e Information communication system

1. An information communication system comprising:
a detection unit that detects vital information of a user;
an acquisition unit that acquires, online, the vital information detected by the detection unit; and
a reporting unit that reports information related to the vital information acquired by the acquisition unit to a related-person terminal held by a predetermined related person.

2. The information communication system according to claim 1, further comprising an online medical care unit that executes a process for performing online medical care on the user when the information related to the vital information is reported to the related-person terminal by the reporting unit in response to a fact that the vital information satisfies a predetermined condition.

3. The information communication system according to claim 1, comprising:
a storage unit that stores the vital information detected by the user in a terminal;
an abnormality determination unit that detects an abnormality in the vital information; and
a transmission unit that conveys the information stored in the terminal to an online server, wherein
the reporting unit reports, to the predetermined related person, a fact that the abnormality in the vital information of the user is determined.

4. The information communication system according to claim 3, wherein
the acquisition unit acquires patient data including at least one selected from information related to an amount of insulin injection, the number of insulin injections, and meals of the user, and
the reporting unit reports, to the user, at least one selected from insulin doses and meals reflecting nutrient balance based on the patient data.

5. The information communication system according to claim 1, further comprising a threshold setting unit that sets an abnormality determination threshold based on past transition of the vital information of the user, wherein the reporting unit reports, to the predetermined related person, a fact that the vital information exceeds the threshold for the user.

6. The information communication system according to claim 1, wherein the detection unit detects the vital information of the user during a web conference.

7. The information communication system according to claim 1, further comprising an emotion estimation unit that estimates an emotion of the user based on the vital information.

8. The information communication system according to claim 1, further comprising:
a reception unit that receives, from the user, reservation information indicating a desire to receive a medical examination; and
a hospital visit reservation unit that registers the reservation information with a predetermined electronic medical record system through a predetermined communication unit in response to reception of the reservation information without going through an Internet.

9. A user terminal comprising:
a detection unit that detects vital information of a user;
a storage unit that records, offline, target information including at least one selected from the vital information, and information related to an amount injected, the number of injections, and meals of the user; and
a transmission unit that transmits the target information to a server during an online time period.

10. A computer control method comprising:
a detection step of detecting vital information of a user;
an acquisition step of acquiring, online, the vital information detected in the detection step; and
a reporting step of reporting information related to the vital information acquired in the acquisition step to a related-person terminal held by a predetermined related person.

11. A non-transitory computer readable medium storing a computer program causing a computer to execute:
a detection step of detecting vital information of a user;
an acquisition step of acquiring, online, the vital information detected in the detection step; and
a reporting step of reporting information related to the vital information acquired in the acquisition step to a related-person terminal held by a predetermined related person.
